(19) United States
(12) Patent Application Publication
(10) Pub. No.: US 2012/0271310 A1
(43) Pub. Date: Oct. 25, 2012

(54) Volar plate device and operative technique

(76) Inventor: Tracy Scott McGee, Leesburg, GA (US)

(21) Appl. No.: 13/066,732

(22) Filed: Apr. 22, 2011

(51) Int. Cl. A61B 17/80

(52) U.S. Cl. 606/71; 606/280; 606/286

(57) ABSTRACT

To establish the correct volar angle between the proximal radius and a distal fragment of the radius a cortical half pin with a self-threading screw is mounted in a collet. The collet is supported on a pair of members that clamp a volar plate under the collet, the volar plate has a hole in alignment with the half pin that enables the plate to pivot in a plane parallel to the members. The volar plate is tilted to contact the distal fragment and to be joined to the fragment. The half pin is screwed into the proximal radius and the members and collet are removed in order to allow the volar plate to be pressed back against the proximal radius and secured to the radius, thus establishing the correct volar tilt for the distal fragment relative to the proximal position of the radius.

### CROSS REFERENCE TO RELATED APPLICATION

None.

### FEDERALLY SPONSORED RESEARCH OR DEVELOPMENT

None

### NAMES OF PARTNERS TO JOINT RESEARCH AGREEMENT

None

### REFERENCE TO “SEQUENCE LISTING”

None

### BACKGROUND

This invention relates to methods and apparatus for tilting a volar plate to match the misalignment of a broken distal fragment of the radius with the angle of the volar plate and, on joining the distal fragment to the volar plate, removing the tilting apparatus and pressing the volar plate against the proximal portion of the radius to align the fracture with a normal volar tilt relative to the proximal portion of the radius, and the like.

The shorter and thicker of the two bones in the forearm, on the same side as the thumb is known, technically, as the radius. That portion of the radius at the end of the bone opposite to the elbow, that is, the distal part of the radius, when it is a broken fragment requires special treatment to restore the injured arm to an approximation of its former utility.

This treatment frequently involves securing a plate on the palm side of the radius, a volar plate, to the proximal portion of the radius, that is the part of the bone between the elbow and the distal fragment.

The volar plate is then secured to the distal fragment and the plate remains permanently in place, joining together the proximal bone and the distal fragment.

There is a great deal of difficulty, however, in aligning the distal fragment in all planes with the corresponding end of the proximal radius in a manner that restores the arm almost to its full use. This is particularly difficult with respect to adapting the angular orientation of the distal fragment to the correct volar tilt relative to the proximal radius. Thus far, volar plates are incapable of meeting this need.

### BRIEF SUMMARY OF THE INVENTION

The problem of matching the alignment in all planes of the distal radius fragment with the corresponding volar tilt relative to the proximal radius are overcome, to a great extent through the practice of the invention

Illustratively, a pair of “C” shaped members each clamp an opposite edge of a volar plate. The volar plate, moreover, has a lengthwise slot formed in its mid-section to enable the threaded end of a “half pin” to protrude between the members, through the lengthwise slot and into a prepared hole in the proximal radius. Part of this half pin protrudes over the members, on the side away from the radius, and is received in a collet that is mounted on the members in a manner that permits the collet to pivot in a plane parallel with and between the two members. The collet has an internal thread that engages a threaded stem, the stem clamping the end of the half pin that protrudes over the members to permit the stem to screw the threaded end of the half pin into the proximal radius.

The volar plate, consequently, can be tilted relative to the proximal radius through an angle limited only by the size of the lengthwise slot in order to match the angular orientation of the distal radial fragment. When so matched, that volar plate is secured to the radial fragment and the members are removed from contact with the volar plate. In turn, the volar plate now is free to be pressed against the proximal radius and fixed in place to the proximal radius.

So treated, the angular orientation of the distal fragment establishes a normal volar tilt with respect to the proximal radius. Thus, the invention provides a significantly improved basis for restoring the patient almost to the full use of the injured arm.

These, and other advantages of the invention will be understood through a reading of the following detailed description of preferred embodiments of the apparatus taken with the figures of the Drawing. The scope of the invention, however, is defined only through the claims appended hereto.

### BRIEF DESCRIPTION OF THE SEVERAL VIEWS OF THE DRAWING

FIG. 1 is a perspective view of a typical embodiment of the invention;

FIG. 2 is a plan view of a volar plate for use with the embodiment of the invention shown in FIG. 1;

FIG. 3 is a side elevation of the embodiment of the invention shown in FIG. 1;

FIG. 4 is a side elevation of a radius with the volar plate shown in FIG. 2 secured to the proximal radius and the distal fragment at the completion of treatment; and

FIG. 5 is a perspective view in full section of another embodiment of the invention.

### DETAILED DESCRIPTION OF THE PREFERRED EMBODIMENTS OF THE INVENTION

For a more complete appreciation of the invention, attention is invited to FIG. 2 which shows a volar plate 10, of metal, plastic, or other suitable material that is compatible with the human body and sufficiently strong to bear those loads encountered during the use of the human arm (not shown). As illustrated, the volar plate 10 has a generally “T” shaped configuration. Holes 11, 12 are formed in a proximal shank 13 of the plate 10 and in accordance with a feature of the invention a lengthwise slot 14 also is formed in the plate 10. The slot 14, moreover, is in alignment with longitudinal axis 15 of the shank 13.

Transverse to the axis 15 and at the distal end of the plate 10 is a distal portion 16 of the plate 10 that accommodates two sets of holes. Two smaller diameter holes 17, 20, each to accept a wire (not shown in FIG. 2) are the first set of holes. Six larger diameter holes 21 through 26, sufficient to accommodate suitable screws or pins also are formed in the distal portion 16 of the plate 10 as the second set.

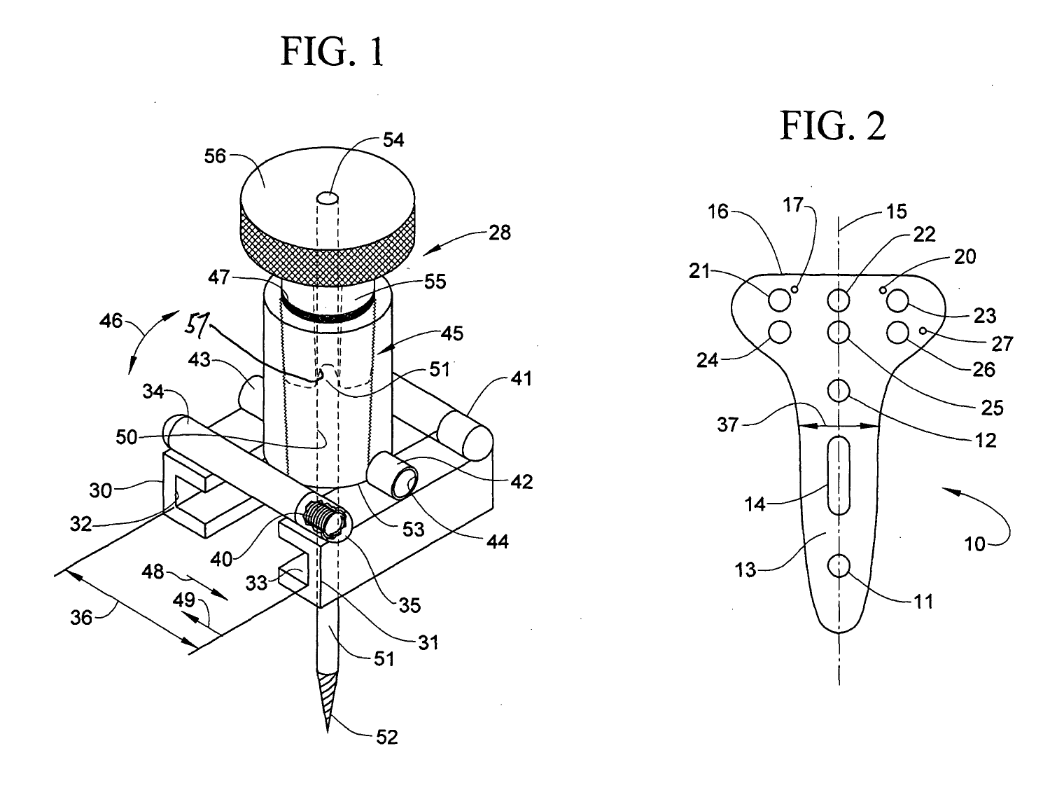

Turning now to FIG. 1 a pair of generally “C” shaped lengthwise members 30, 31 in tilting structure 28 are positioned with respective concavities 32, 33 facing each other. A spring biased barrel 34 is mounted on the member 30 to extend across gap 36 between the concavities 32, 33 in the respective members 30, 31. The gap 36 is equal to width 37 (FIG. 2) of the proximal shank 13 of the plate 10. The barrel 34 is received in a cap 35 that is secured on the member 31, opposite to and in alignment with the barrel 34. The spring biased barrel 34 with the cap 35 is characterized by defining two stops, the first stop establishes the width of the gap 36 to enable the opposing concavities 32, 33 to clamp and hold the proximal shank 13 (FIG. 2) of the plate 10 securely between the members 30, 31 (FIG. 1).

The second stop fixed by the barrel 34 and cap 35, under the force of biasing spring 40, releases the plate 10 from being gripped between the opposing concavities 32, 33. Second stop width between the members 30, 31, as a minimum, must be sufficiently greater than the shank width 37 (FIG. 2) to permit the members 30, 31 (FIG. 1) both to receive the shank 13 within the concavities 32, 33 and to separate the members 30, 31 from the plate at the end of the surgical procedure.

At the ends of the members 30, 31 opposite to and on the same side of the members 30, 31 as the barrel 34 and the cap 35 is a similar barrel and cap combination 41.

Essentially at the midpoint of the members 30, 31 and on the same side of the member 30, 31 as the barrel and cap combination 41 a pair of journals 42, 43 are mounted on the members 31, 30, respectively. A shaft 44, seated in the journal 42 is secured (not shown in the drawing) to the outer surface of a cylindrical collet 45. A second shaft (also not shown in the drawing) is secured to the outer surface of the collet 45 and on a side of the collet 45 that is opposite to the place of attachment for the shaft 44. Both of these shafts permit the collet 45 to pivot in a plane that is parallel to the gap 36 between the members 30, 31 as illustrated through arrow 46. Further in this respect the combined length of the two shafts and the outer diameter of the collet 45 are substantially greater that the width 37 (FIG. 2) of the plate 10 to enable the members 30, 31 to clamp and release the volar plate 10 through movement in the directions of arrows 48, 49.

As illustrated in FIG. 1, the collet 45 has an inwardly tapered threaded central bore 47 through about half the depth of the collet 45. A smaller diameter bore 50 is in alignment with the threaded bore 47. A half pin 51 preferably 3.5 mm, is received in the smaller diameter bore 50. A self-threading cortical screw 52 on the end of the pin 51 protrudes from end 53 of the collet to extend beyond the members 30, 31 and to pivot selectively with the collet 45 in the directions of the arrow 46. It should be noted that beyond the self-threading screw 52 on the half pin 51, the surface of the half pin 51 is smooth and cylindrical.

Opposite end 54 of the half pin 51 extends not only through the threaded central bore 47 of the collet 45, but also through threaded stem 55 and its associated knurled knob 56, located outside of the collet 45. As shown, the threading on the stem 55 encloses a portion of the half pin 51. An inverted “V’ shaped split 57 also on the stem 55 encloses a portion of the half pin 51 such that as the stem 55 is screwed toward the end of the inwardly tapered and threaded central bore 47, the “V” shaped split moves radially inwardly to enable the stem 55 to clutch the enclosed smooth, cylindrical portion of the half pin 51 and compel the half pin 51 to rotate with the stem 55.

To rotate the stem 55 and the half pin 51, the stem 55 protrudes beyond the collet 45 and is joined to the knurled knob 56.

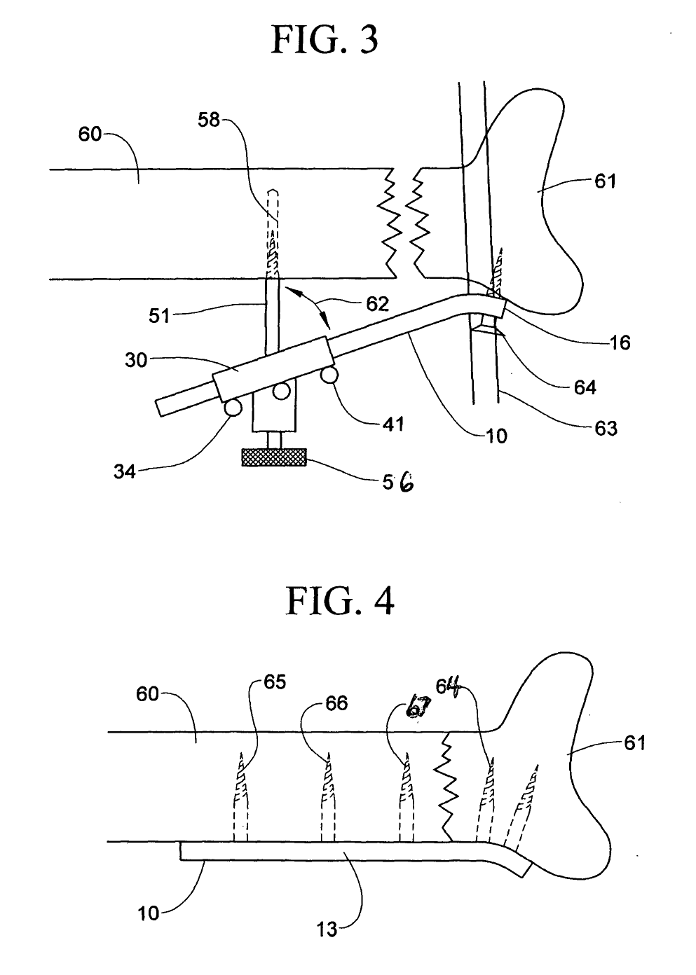

In operation, and as best shown in FIG. 3, the volar plate 10 is clutched in the concavities 32, 33 (FIG. 1) formed by the members 30, 31 (only the member 30 is shown in FIG. 4). To press together the members 30, 31 the barrel 34 with the barrel and cap combination 41 are engaged in the first stop of their two-stop positions.

The half pin 51 is placed over exposed radius 60 and is received in a prepared 2.7 mm bore 58. By rotating the knob 56, the self-threading cortical screw 52 on the half pin 51 threads itself into the structure of the smaller diameter bore 58 of the radius 60.

To tilt the volar plate 10 in order to join the plate 10 to distal fragment 61 the plate 10 in accordance with a feature of the invention, is pivoted through an appropriate angle 62 to enable the distal portion 16 of the volar plate to bear against the distal fragment 61 of the radius 60.

Wires 63 are passed through the wire holes 17, 20 (FIG. 2); and screws 64 are passed through the larger holes 21 through 26 in the volar plate 10, all to join the distal fragment 61 (FIG. 3) to the distal portion 16 of the plate 10.

In passing, it should be noted that the maximum angle 62 through which the volar plate 10 can be titled is limited only through the length of the longitudinal slot 14 (FIG. 2) in the volar plate 10. Thus, when tilting the members 30, 31 (FIG. 1) and the accompanying plate 10 are tilted through an appropriate angle illustrated by the arrow 46, the sweep or pivot range of the volar plate 10 is stopped only when either of the longitudinal ends of the slot 14 contact the half pin 51.

Returning to the operational procedure, once the distal fragment 61 (FIG. 3) is joined to the distal portion 16 of the volar plate 10, the barrel 34 and the barrel and cap combination 41 are released to the second stop, in which the concavities 32, 33 (FIG. 1) in the respective members 30, 31 release their respective grips on the proximal shank 13 (FIG. 2) of the volar plate 10. By turning the knob 56 in a direction that releases the engagement between the threaded stem 55 and the half pin 51 the tilting structure 20 is completely withdrawn from the volar plate.

To complete the procedure, the volar plate 10 (FIG. 4) is pressed against the exposed proximal radius 60 and, in so doing, the distal fragment 61 necessarily is drawn into the correct orientation relative to the corresponding end of the proximal radius 60. Self-threading screws 65, 66, 67 are applied through respective holes in the proximal shank 13 to complete the procedure in which the distal fragment 61 has the correct angle of volar tilt, to provide a better basis for returning the injured arm almost to full utility

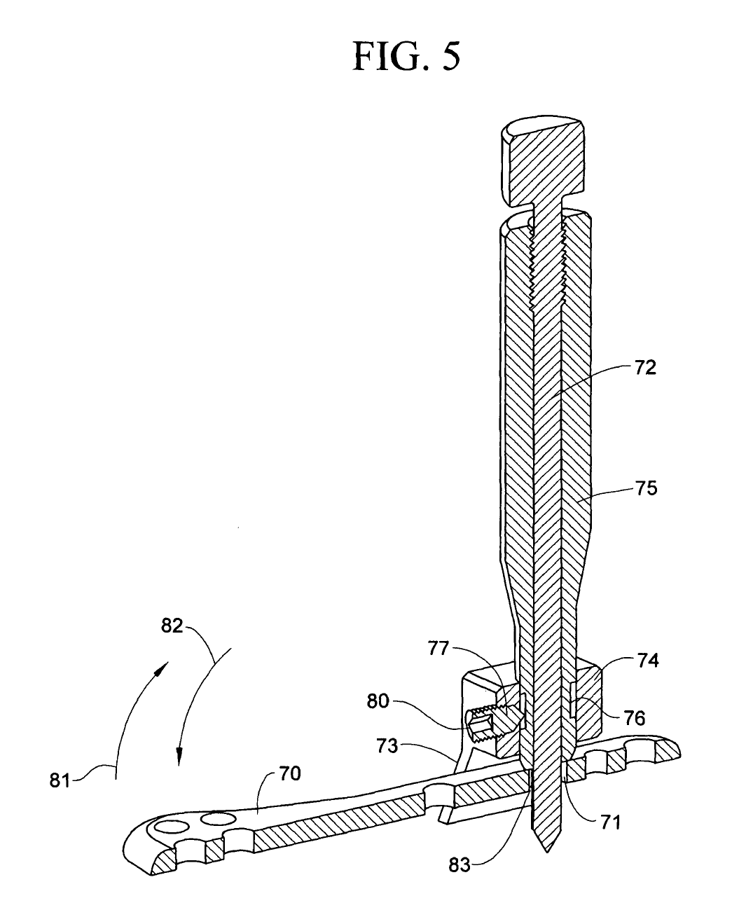

Attention now is invited to FIG. 5 which shows a further embodiment of the invention. As illustrated volar plate 70 has a hole 71 that is somewhat larger than the corresponding diameter of a pin 72. The pin 72 also extends toward an exposed proximal radius (not shown in FIG. 5). Two members 73 (only member 73 is shown in the drawing) are slightly skewed relative to the pin 72 in order to stabilize the volar plate 70 between the two members. A bridge 74 extends across the plate 70 and supports both of the members (only member 73 is shown in FIG. 5) and a collet 75. Within the bridge 74, the collet 75 has an annular ring 76 to provide a bearing surface for a stabilizing set screw 77 that is received in a mating, threaded hole 80 formed in the bridge 74.

In operation, the volar plate 70 can be tilted in the directions of arrows 81, 82, the sweep of the tilt being limited by the width of gap 83 between the surface of the hole 71 and the opposing surface of the pin 72. Accordingly, the volar plate 70 is joined to the distal fragment in the manner described above, the set screw 77 is withdrawn from contact with the annular ring 76, the pin 72, bridge 74 and the members 73, moreover, are withdrawn from contact with the volar plate 10.

Pressing the volar plate 70 against the exposed proximal radius carries the distal fragment into a proper angular relation with the proximal portion of the radius. The volar plate 70, in the embodiment of the invention shown in FIG. 5, however, can only be lifted by about 5 mm above the radius.

Thus, in accordance with the principles of the invention, an improved surgical device and technique now offer a superior basis for restoring a broken radius nearly to its normal function.

1. Structure for matching the alignment of a distal radius fragment to the proximal radius comprising, a volar plate having a distal portion and a lengthwise proximal portion and having a hole formed in said proximal portion, a pair of members each of said members engaging at least a part of the respective sides of said volar plate proximal portion, a collet between said members and in alignment with said volar plate hole, a half pin within said collet, and protruding through said volar plate hole, said volar plate being able to pivot relative to said half pin within said proximal portion hole in a direction parallel with said lengthwise proximal portion in order to match the tilt of said volar plate to the distal fragment.

2. A structure according to claim 1 further comprising a bridge supporting said members, said members being skewed relative to each other, and a set screw received in said bridge to stabilize said collet in said bridge.

3. A structure according to claim 1 wherein said proximal portion hole further comprises a lengthwise slot, said pair of members being generally parallel with each other and said members having opposed concavities for selectively clamping and releasing said respective volar plate sides.

4. A structure according to claim 1 further comprising a journal on each of said members, and a shaft in each of said journals, each of said shafts being connected to opposite sides of said collet to enable said collet to pivot in a plane parallel with said members.

5. A structure according to claim 1 further comprising a pair of barrel and cap combinations, each mounted on said members on opposite sides of said collet, said barrel and cap combinations each having two positions for selectively drawing said members together to clamp said volar plate and separating said members to release said volar plate.

6. A structure according to claim 4 wherein said collet has a centrally disposed hole, an internal thread formed in a portion of said hole, a threaded stem protruding into said internally threaded portion of said collet hole, said half pin extending into said internally threaded stem and selectively clutched by said stem for rotation with said stem.

7. (canceled)

8. A method of matching the volar tilt of a distal radius fragment with the proximal radius comprising the steps of clamping a volar plate to a pivot, protruding a half pin from said pivot through an oblong hole in said volar plate, securing said half pin in the proximal radius, tilting said pivoted volar plate to match the distal radial fragment, securing said distal fragment to said volar plate, removing said pivot, pressing said volar plate to the proximal radius and securing said volar plate to the proximal radius.
